(19) United States
(12) Patent Application Publication
(10) Pub. No.: US 2016/0115539 A1
(43) Pub. Date: Apr. 28, 2016

(54) USE OF A SLC MAMMALIAN SKIN TRANSPORTER

(71) Applicant: GALDERMA RESEARCH & DEVELOPMENT, Biot (FR)

(72) Inventor: Hanan OSMAN-PONCHET, Antibes (FR)

(21) Appl. No.: 14/891,573

(22) PCT Filed: May 14, 2014
(86) PCT No.: PCT/EP2014/059887

§ 371 Date: Nov. 16, 2015

Related U.S. Application Data
(60) Provisional application No. 61/823,756, filed on May 15, 2013.

(51) Int. Cl. C12Q 1/68; A61K 9/00; A61K 31/496

(52) U.S. Cl. CPC C12Q 1/6883; C12Q 1/6886; A61K 31/496; A61K 9/0014; C12Q 2600/158; C12Q 2600/136; C12Q 2600/166

(57) ABSTRACT

A method of using a Solute Carrier (SLC) mammalian skin transporter is described for evaluating the penetration of topical drug. Also described, are methods of using the transporter to diagnose skin diseases and to treat skin diseases.

The described methods may be useful for identifying topically applied drugs for treatment of skin diseases or as biomarkers for skin diseases. The described methods are intended to assist in characterizing and analyzing the regulation of the expression of solute carrier transporters in mammalian skin.

### BACKGROUND

1. Technical Field of the Invention

The present invention relates to the use of Solute Carrier (SLC) mammalian skin transporter to evaluate penetration of topical drug, to diagnostic skin diseases and to treat skin diseases.

The invention will find application especially for identifying topically applied drugs for treatment of skin diseases or as biomarkers for skin diseases. The invention aims at characterize and analyze the regulation of the expression of solute carrier transporters in mammalian skin.

2. Related Art of the Invention

Most identified drug transporters belong to ATP-binding Cassette (ABC) and Solute Carrier (SLC). Recent research indicates that these transporters play an important role in the absorption, distribution and excretion of drugs, and are involved in clinically relevant drug-drug interactions.

The solute carrier (SLC) proteins constitute a group of membrane-integrated transporters. Solutes that are transported by the various SLC group members are extraordinarily diverse and include both charged and uncharged organic molecules as well as inorganic ions. Members of the SLC group could belong to facilitative transporters, allowing solutes to flow passively with their electrochemical gradients, or to secondary active transporters allowing the solute to flow against its electrochemical gradient by having the transport coupled to another solute that passively flows with its electrochemical gradient such that the free energy change for the two solutes together is still favorable.

Drug transporter-based interactions have been well documented in liver and kidney for systemic drugs. However very little is known about the role of drug transporters in human skin in the disposition of topically applied drugs, especially SLC transporters.

The expression profile of SLC transporters is well documented in liver and kidney. However, in human skin, the expression profile of SLC transporters, particularly MATE (Multidrug and toxin extrusion) transporters are poorly documented.

There is a need of tools to evaluate the penetration in mammalian skin of drugs applied topically and to diagnostic and treat skin diseases.

### SUMMARY OF THE INVENTION

Surprisingly, the inventor found that the characterization of the expression profile of SLC transporters in human skin, the comparison with the liver's and kidney's expression profile and the study of the regulation of SLC transporters by Rifampicin and UV radiation will give rise to tools to meet the current needs.

The present invention relates to the use of mammalian skin SLC transporters. SLC transporters detected in the mammalian skin have shown to influence the penetration of topically applied drugs. In addition, the expression of detected SLC transporters is influenced by environmental factors and some xenobiotics.

A first aspect of the invention relates to the use of mammalian skin SLC transporters to evaluate the penetration of topically applied drugs in mammalian skin.

Another aspect of the invention relates to the use of mammalian skin SLC transporters to diagnostic and to treat skin diseases.

### BRIEF EXPLANATION OF THE DRAWINGS

The purposes, objects, characteristics and advantages of the invention will better emerge from the detailed description of an embodiment thereof which is illustrated by the following accompanying figures in which:

FIGS. 1 and 1b is: Organoculture of human skin samples.

FIG. 2: Expression profile of SLC transporters in human skin samples from 3 different donors.

FIG. 3: Expression profile of SLC transporters in human hepatocytes (pool of 26 donors).

FIG. 4: Expression profile of SLC transporters in human kidney total RNA (2 donors).

FIG. 5: Comparison of expression of SLCO4A1 in human skin, hepatocytes and kidney.

FIG. 6: Effect of Rifampicin on mRNA expression of SLC47A1 and SLC47A2 in human skin.

FIG. 7: Effect of Rifampicin on mRNA expression of SLC47A1 in human hepatocytes

FIG. 8: Effect of solar simulator (UV radiation) on expression of SLC transporters in ex vivo human skin.

### PREFERRED EMBODIMENTS

Optional characteristics which may be used in association or alternately are given hereinafter prior to making a detailed list of the embodiments of the invention.

According to one aspect of the invention, mammalian skin SLC transporter can be used to evaluate topical drug exposure in mammalian skin.

For example, a method of evaluating topical drug exposure in mammalian skin comprises measuring at least one of gene expression level and expression profile of an SLC transporter in the skin and correlating at least one of the measured gene expression level and expression profile to a level of topical drug exposure in the skin. This method can advantageously further comprises a step of adjusting administration of topical drug in view of the based on the level of topical drug exposure.

Advantageously, the SLC transporter is selected from the group consisting of SLC47A1, SLC47A2, SLCO3A1, SLCO2B1, SLCO4A1 and combinations thereof.

According to one aspect of the invention, mammalian skin SLC transporter can be used to evaluate drug-drug interactions in mammalian skin. For example, a method of evaluating drug-drug interactions in mammalian skin comprises measuring at least one of gene expression level and expression profile of an SLC transporter in the skin and correlating at least one of the measured gene expression and expression profile to a level of drug-drug interaction in the skin. This method can advantageously further comprises a step of adjusting the administration of drug based on the level of drug-drug interaction.

Advantageously, the SLC transporter is selected from the group consisting of SLC47A1, SLC47A2, SLCO3A1, SLCO2B1, SLCO4A1 and combinations thereof

According to one aspect of the invention mammalian skin SLC transporter can be used as a biomarker for skin cancer.

Advantageously, skin cancer is induced by solar exposure.

Advantageously, skin cancer is selected among the list consisting of actinic keratosis, basal cell carcinoma, melanoma, Kaposi's sarcoma (KS), squamous cell carcinoma and combinations thereof.

Advantageously, the SLC transporter is selected from the group consisting of SLC47A1, SLC47A2, SLCO3A1, SLCO2B1, SLCO4A1 and combinations thereof

For example, a method of diagnosing skin cancer in mammalian skin comprises measuring at least one of gene expression level and expression profile of an SLC transporter in the skin as a biomarker for skin cancer and correlating at least one of the measured gene expression and expression profile to a diagnosis of skin cancer in the skin.

The method further comprises administering an anti-cancer treatment to the skin based on the skin cancer diagnosis.

According to one aspect of the invention, mammalian skin SLC transporter can be used to identify an active agent for treatment of skin cancer. For example, a method of identifying an active agent for treating skin cancer in mammalian skin comprises administering an active agent to the skin and measuring at least one of gene expression level and expression profile of an SLC transporter in the skin and correlating at least one of the measured gene expression and expression profile with a determination of whether the active agent is useful for treating skin cancer. Advantageously, the SLC transporter is selected from the group consisting of SLC47A1, SLC47A2, SLCO3A1, SLCO2B1, SLCO4A1 and combinations thereof

Accordingly to another aspect of the invention, a method of evaluating a drug for the regulation of SLC transporters, the method comprising contacting the drug with mammalian skin and obtaining an expression profile of SLC transporters expressed by the skin to determine whether the drug regulates the expression of SLC transporters. Advantageously, the SLC transporter is selected from the group consisting of SLC47A1, SLC47A2, SLCO3A1, SLCO2B1, SLCO4A1 and combinations thereof

Accordingly to another aspect of the invention, a method of evaluating a drug for the treatment of skin cancer, the method comprising contacting the drug with cancerous mammalian skin cells, obtaining an expression profile of SLC transporters expressed by the skin cells, comparing the expression profile with a control expression profile, wherein a difference between the expression profile and the control expression profile indicates that the drug is potentially useful for the treatment of skin cancer.

Advantageously, the control expression profile is that of SLC transporters in cancerous mammalian skin that were not contacted with the drug.

Advantageously, the SLC transporter is selected from the group consisting of SLC47A1, SLC47A2, SLCO3A1, SLCO2B1, SLCO4A1 and combinations thereof

As mentioned above, little information is known regarding the transporters present in the skin especially concerning the SLC transporters. It is now an important subject as medicament agencies start to require details on the influence of new systemic drugs on the drug transporters (drug as a substrate or as an inhibitor of transporters).

Moreover, some SLC transporters including multidrug and toxin extrusion (MATE) transporters of the SLC47 family have been identified as efflux transporters in liver and kidney for xenobiotics including several clinically used drugs such as the anticancer drug oxaliplatin. Thus, their expression levels and pattern could be of relevance for treatment of skin cancer like actinic keratosis, basal cell carcinoma, melanoma, Kaposi's sarcoma (KS) and squamous cell carcinoma.

The invention characterizes the expression profile of SLC transporter in mammalian skin. Mammalian skin includes human skin and skin of mammalian animals.

The use of mammalian skin SLC transporters to evaluate the penetration of topically applied drugs in mammalian skin includes the evaluation of the topical drug exposure and the evaluation of drug-drug interactions.

The use of mammalian skin SLC transporters to diagnostic and treat skin disease include the use as biomarkers for skin cancer and the use to identify an active agent for treatment of skin cancer.

For the preparation of ex vivo human skin samples, the skin is obtained from plastic surgery. Skin samples originate from male as well as from female. They can be obtained from abdominal or mammary tissues, but also from other anatomical site of the body. Fresh or frozen skin samples may be used. Preferably fresh skin samples are used as the activity of SLC transporters and others enzymes are better preserved. By fresh skin samples, we understand skin samples obtained just after skin excision up to about 24 hours following excision, preferably about 4 hours after excision.

Alternatively, skin samples may be maintained in organoculture conditions up to about 72 hours after excision and before the evaluation method of SLC transporter implication starts.

Gene expression of eleven SLC transporters (Table 1) was measured by TaqMan Real-time RT-PCR in human skin, kidney and hepatocytes. GAPDH was used as housekeeping gene. Detailed method is given at example 1.

TABLE 1
List of SLC transporters investigated

| Gene | Transporter |
|---|---|
| SLCO1B1 | OATP1B1 |
| SLCO1B3 | OATP1B3 |
| SLCO2B1 | OATPB |
| SLCO3A1 | OATPD |
| SLCO4A1 | OATPE |
| SLC22A1 | OCT1 |
| SLC22A2 | OCT2 |
| SLC22A6 | OAT1 |
| SLC22A8 | OAT3 |
| SLC47A1 | MATE1 |
| SLC47A2 | MATE2 |

There are five common types of skin diseases:

Inflammatory Skin Diseases

These include eczema, dermatitis, psoriasis, diaper rash and acne. Some of these skin diseases can last for extended periods of time. Topical ointments are usually prescribed to lessen the itching and swelling.

Viral Skin Diseases

These include chicken pox, measles, herpes 1, herpes 2, and shingles. Topical and prescription medications are available for viral skin diseases.

Fungal Skin Diseases

Microscopic fungi are the cause of fungal infections. Candida, athlete's foot and ringworm are all fungal infections. Treatments can include oral medications, topical ointments, powders, and oral antiseptics.

Bacterial Skin Diseases

These include impetigo, cellulitis, MRSA, folliculitis, scabies, and necrotizing fasciitis are all bacterial skin diseases. Treatment may involve antibiotics, draining the area, or in extreme cases, removing the infected area.

Cancerous Skin Diseases

Basal cell cancer, squamous cell cancer, and melanoma are types of cancerous skin diseases.

Examples of topically applied drugs used for the treatment of skin diseases are listed below:
Acne: Tretinoin, benzoyl peroxide, clindamycin, doxycycline, isotretinoin, tetracycline, minocycline, salicylic acid, azelaic acid, erythromycin topical, drospirenone-ethinyl estradiol, tazarotene, benzoyl peroxide-clindamycin, ethinyl estradiol-norgestimate, sulfacetamide sodium, or clindamycin-tretinoin.
Psoriasis: Cortisone, retinoids derived from vitamin A, vitamin D analogues, salicylic and lactic acid.
Rosacea: Metronidazole and azelaic acid.
Vitiligo: Hydroquinone
Impetigo: Erythromycin and mupirocin.

Several skin cancers are induced by solar exposure. Such skin cancer can be actinic keratosis, basal cell carcinoma, melanoma, Kaposi's sarcoma (KS), squamous cell carcinoma and combinations thereof.

The following examples are provided merely to as illustrative of various aspects of the invention and shall not be construed to limit the invention in any way.

### Example 1

### Skin Organoculture

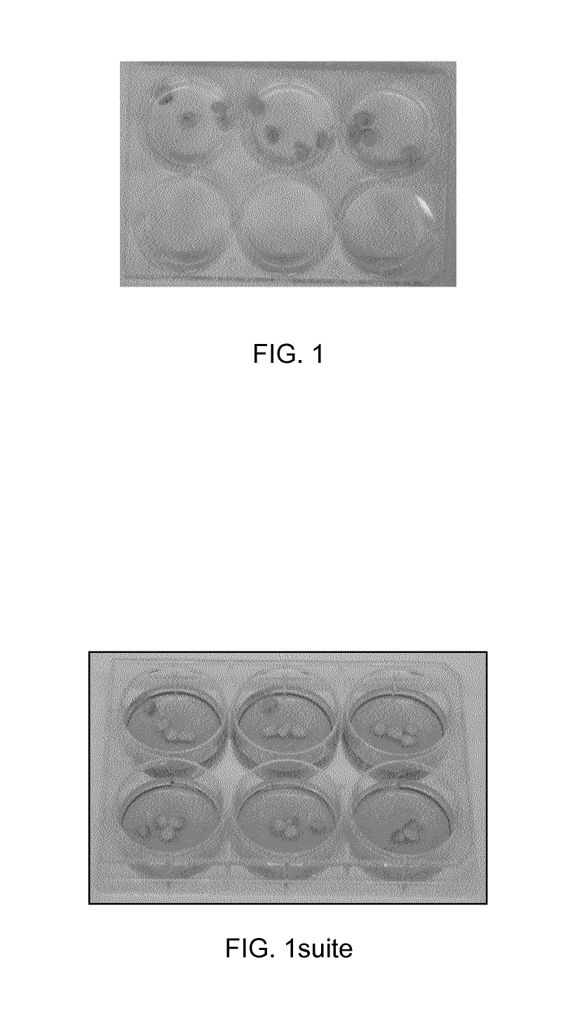

Fresh human skin samples from 3 different donors were used and maintained in organoculture. Four skin biopsies of 6 mm diameter were used per well of 6-well plate, filled with long term skin culture medium (Biopredic, France) (FIG. 1). Skin samples were treated with 20 μM Rifampicin during 3 days. In a separate experiment, skin samples were exposed during one hour every day during 3 days to UV lights via a solar simulator (UVA 110 W/m²; UVB 20 W/m²). Untreated skin samples were used as control. The culture plates were kept in a cell culture incubator set at 37° C., 5% CO₂ and saturated hygrometry. At the end of treatment period gene expression of SLC transporters was measured as described above.

### Example 2

### Gene Expression Analysis by Real Time PCR

After homogenization of skin samples or hepatocytes in lysis buffer (Promega), total RNA was isolated using SV Total RNA Isolation System (Promega), in accordance with the instructions provided by the constructor. RNA concentrations were quantified spectrophotometrically. Quantification of mRNA expression of human SLC transporters was performed using TaqMan PCR techniques (Applied Biosystems). Experiments were carried out on a 7500 real time PCR System (Applied Biosystems) using Assay-on-Demand gene expression products. For this, 500 ng of total RNA were reverse-transcribed using the High Capacity RNA to cDNA Master Mix kit (Applied Biosystems). PCR amplifications were performed in a total volume of 25 μL using the TaqMan Universal Master Mix (Applied Biosystems). Denaturation was performed at 95° C. for 10 min, followed by 40 PCR cycles with the following specifications: 95° C. for 15 s and 60° C. for 60 s. Glyceraldehyde-3-phosphate dehydrogenase (GAPDH) was used as a reference gene for normalization in each sample. TaqMan Gene Expression Assays from Applied Biosystems were used in the expression profiling experiments. All RT-PCR measurements were performed in triplicate.

Relative quantification of the expression level of each transcript in each sample was calculated using the comparative threshold cycle (Ct) method, also called delta delta Ct method [Livak K J, Schmittgen T D. Analysis of relative gene expression data using real-time quantitative PCR and the 2(−Delta Delta C (T)) method. Methods 2001; 25:402-8]. Briefly, expression values for target genes were normalized to the concentration of GAPDH, which showed the least variation among reference genes in our biological systems. Gene expression values were calculated based on the comparative threshold cycle (Ct) method, in which RNA samples were designated as calibrators to which the other samples were compared. The Ct data for SLC transporters and GAPDH in each sample were used to create delta Ct values (Ct SLC transporter−Ct GAPDH). Thereafter, delta Ct values were calculated by subtracting the delta Ct of the calibrator from the Ct value of each target. The RQs or fold change were calculated with the following equation: RQ=2^{-delta delta Ct}. The results are expressed as 2^{-delta Ct}, or as 2^{-delta delta Ct}.

### Example 3

### Expression Profile of SLC Transporters in Human Skin

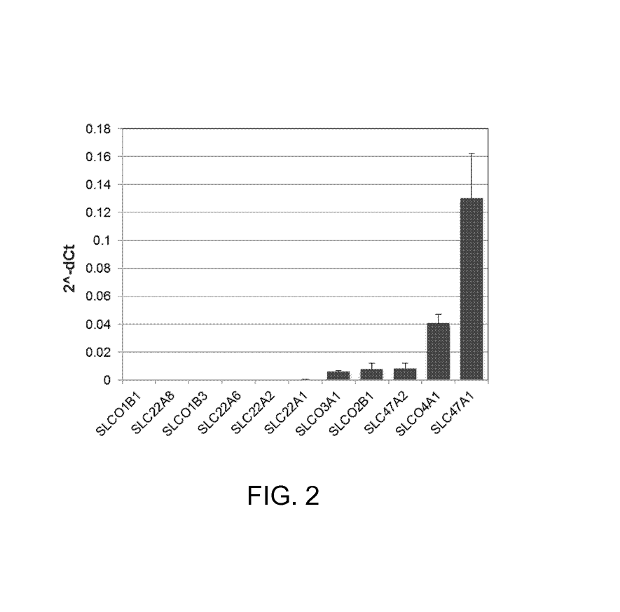

To determine whether the SLC transporter genes are expressed in human skin, mRNA expressions of eleven SLC transporters were measured in human skin from three different donors using real time PCR. The results presented in FIG. 2 show that among the 11 tested transporter genes, 6 were not detected in human skin (OATP1B1, OATP1B3, OCT1, OCT2, OAT1 and OAT3). On the contrary, 5 transporter genes were detected in human skin, those coding for OATPB, OATPD, OATPE, MATE1 and MATE2. The expression of SLC47A1 gene coding for MATE1 transporter was the highest compared to the other genes detected (FIG. 2).

### Example 4

### Comparison of Expression Profile of SLC Transporters in Human Skin, Kidney and Hepatocytes

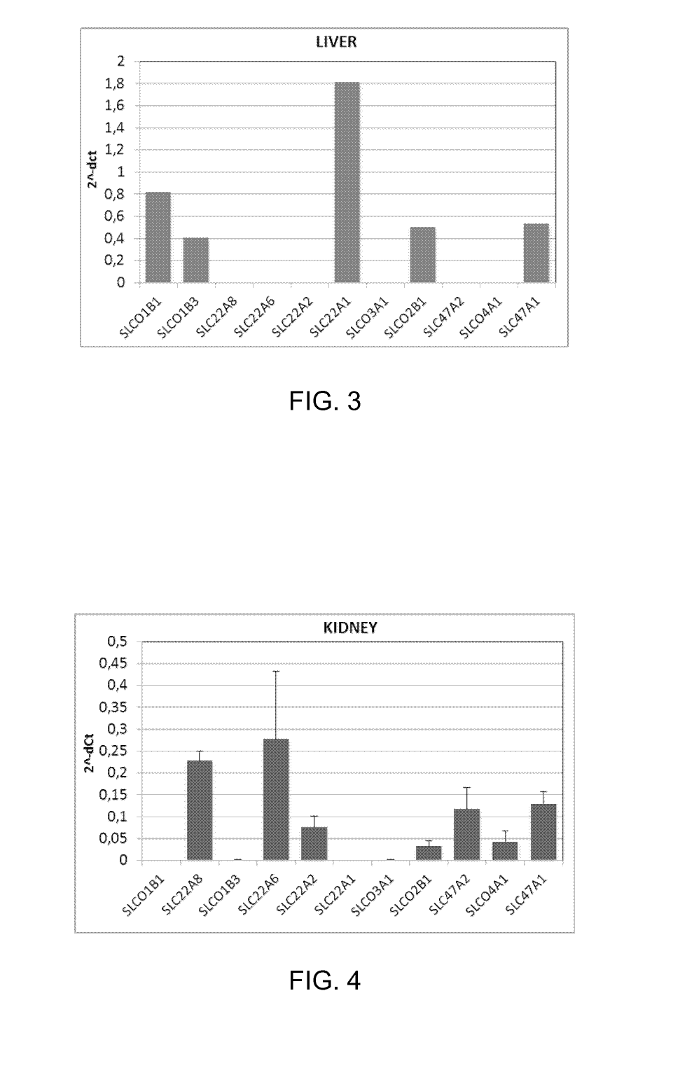

mRNA expression of SLC transporters were analyzed in human cryopreserved hepatocytes (pool of 26 different donors) and in human kidney total RNA samples from 2 different donors. The results presented in FIG. 3 and FIG. 4 show that the expression profiles of SLC transporters are specific to the organ considered.

In hepatocytes, SLC22A1 coding for OCT1 is the most strongly expressed, while in kidney, it is SLC22A6 coding for OAT1. Moreover, OATPE expression in human skin is 70 times higher than in human hepatocytes and as high as in human kidney.

These results show that human skin has a very specific SLC transporters' expression profile compared to liver and kidney. Moreover they provide for the first time evidence that MATE1 transporter is strongly expressed in human skin.

### Example 5

### Regulation of SLC Transporters in Human Skin: Effect of Rifampicin

The effect of rifampicin on the expression of SLC47A1 and SLC47A2 was investigated in human skin and in human hepatocytes in primary culture (two donors). After 72 hours treatment with Rifampicin 20 μM, gene expression of SLC transporters was measured by Real Time PCR.

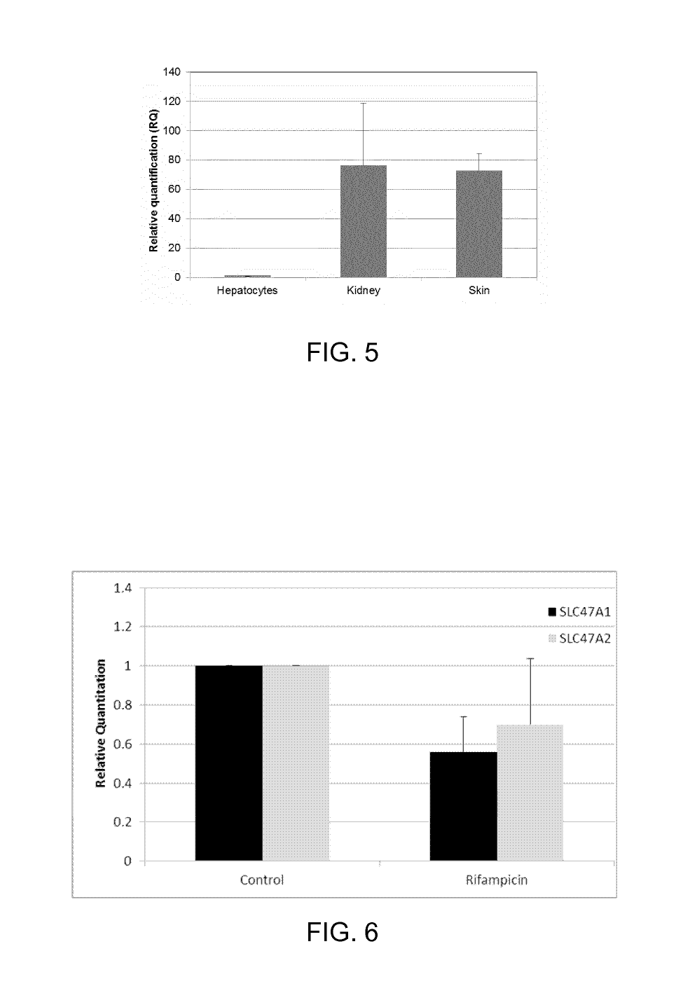

The results show that in human skin (FIG. 5), expression of SLC47A1 (MATE1) and SLC47A2 (MATE2) was strongly diminished after Rifampicin treatment. Indeed, Rifampicin triggers a 44% decrease in MATE1 expression (p≦0.05) and a 30% decrease in MATE2 expression.

Moreover, the results show that the expression of SLC47A1 (MATE1) also importantly decreased (by 48%) in human hepatocytes after being treated with Rifampicin 20 μM for 72 hours (FIG. 6).

Taken together, the results show that the expression of MATE1 and MATE2 transporters is regulated by Rifampicin in human skin and the same phenomenon was observed in fresh human hepatocytes with MATE1.

### Example 6

### Regulation of SLC Transporters in Human Skin: Effect of UV Lights

The effect of UV lights on the expression of SLC transporters was investigated in human skin from two different donors. Human skin biopsies were maintained as explained in the Materials and Methods section and exposed to UV lights for 1 hour every 24 hours using a solar simulator. Control samples were not exposed to UV lights. The expression of the SLC47A1 and SLC47A2 transporters was measured by real time PCR.

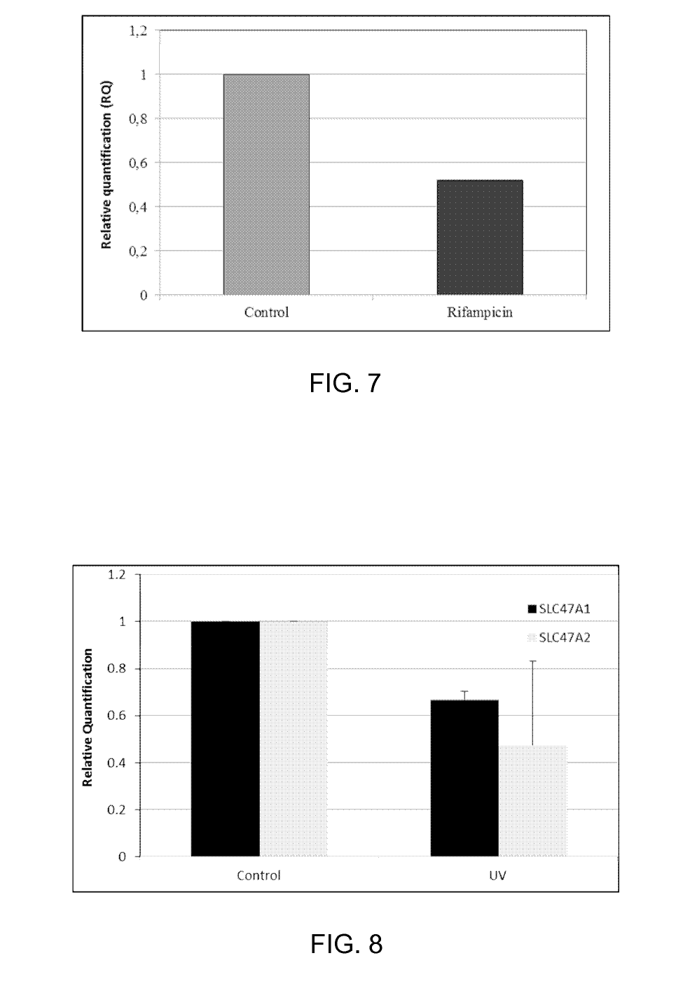

The results presented in FIG. 7 show that following the exposure of human skin to UV lights, the expression of MATE1 and MATE2 importantly decreased by 43% (p≦0.05) and 60%, respectively. This indicates that the exposure of human skin biopsies to UV lights can modulate the expression of some SLC transporters in human skin.

### Discussion

The results show that SLC transporters have a very specific expression profile in human skin (FIG. 2). At least five over the eleven SLC genes studied have been detected in skin, with SLC47A1 (MATE1) being the most expressed.

Expression of SLC transporters in human skin is very different compared to hepatocytes and kidney. Indeed, expression of SLCO4A1 is about 70 times higher in human skin than in human hepatocytes but similar in human kidney (FIGS. 3 and 4).

Moreover, the results show that rifampicin treatment as well as exposure to UV lights down-regulate the expression of SLC47A1 (MATE1) and SLC47A2 (MATE2) in human skin (FIGS. 5, 6 and 7).

### CONCLUSION

The results presented in this work showed that some SLC transporters have a specific expression profile in human skin. For example, expression of MATE1 transporter in human skin is the most important compared to the others SLC transporters, suggesting that MATE1 may play an important role in topical drug exposure and in drug-drug interactions in dermatology.

Furthermore, the extensive expression of MATE1 transporter in human skin, and the down-regulation of its expression by solar simulator exposure suggest that this transporter might serve as biomarkers for skin cancers, particularly those induced by solar exposure. Furthermore, the results suggest that expression of MATE1 transporter in human skin could play an important role in chemosensitivity of cutaneous cancer cells, and thus may be further exploited for the discovery of novel agents for treatment of skin cancers.

This can be confirmed by investigating the expression of MATE1 in skin cancer of different origin. More precisely, by determining mRNA levels of MATE1 in paired cancerous and adjacent non-cancerous specimens from a large number of patients with different types of skin cancer. The results of this experiment will definitively confirm the role of MATE1 in skin cancer.

1.-7. (canceled)

8. A method of evaluating topical drug exposure in mammalian skin, the method comprising measuring at least one of gene expression level and expression profile of an SLC transporter in the skin.

9. The method of claim 8, the method further comprising a step of adjusting administration of topical drug based on the level of topical drug exposure.

10. The method of claim 8, wherein the SLC transporter is selected from the group consisting of SLC47A1, SLC47A2, SLCO3A1, SLCO2B1, SLCO4A1 and combinations thereof.

11. A method of evaluating drug-drug interactions in mammalian skin, the method comprising measuring at least one of gene expression level and expression profile of an SLC transporter in the skin.

12. The method of claim 11, the method further comprising adjusting the administration of drug based on the level of drug-drug interaction.

13. The method of claim 11, wherein the SLC transporter is selected from the group consisting of SLC47A1, SLC47A2, SLCO3A1, SLCO2B1, SLCO4A1 and combinations thereof.

14. A method of diagnosing skin cancer in mammalian skin, the method comprising measuring at least one of gene expression level and expression profile of an SLC transporter in the skin as a biomarker for the skin cancer.

15. The method of claim 14, the method further comprising administering an anti-cancer treatment to the skin based on the skin cancer diagnosis.

16. The method of claim 14, wherein the SLC transporter is selected from the group consisting of SLC47A1, SLC47A2, SLCO3A1, SLCO2B1, SLCO4A1 and combinations thereof.

17. The method of claim 14, wherein the skin cancer is induced by solar exposure.

18. The method of claim 14, wherein the skin cancer is selected from the group consisting of actinic keratosis, basal cell carcinoma, melanoma, Kaposi's sarcoma (KS), squamous cell carcinoma and combinations thereof.

19. A method of identifying an active agent for treating skin cancer in mammalian skin, the method comprising administering an active agent to the skin and measuring at least one of gene expression level and expression profile of an SLC transporter in the skin.

20. The method of claim 19, wherein the SLC transporter is selected from the group consisting of SLC47A1, SLC47A2, SLCO3A1, SLCO2B1, SLCO4A1 and combinations thereof.

21. A method of evaluating a drug for the regulation of SLC transporters, the method comprising contacting the drug with mammalian skin cells and obtaining an expression profile of SLC transporters expressed by the skin cells to determine whether the drug regulates the expression of SLC transporters.

22. A method of evaluating a drug for the treatment of skin cancer, the method comprising contacting the drug with cancerous mammalian skin cells, obtaining an expression profile of SLC transporters expressed by the skin cells, comparing the expression profile with a control expression profile, wherein a difference between the expression profile or the control expression profile indicates that the drug is potentially useful for the treatment of skin cancer.

23. The method of claim 22, wherein the control expression profile is that of SLC transporters in cancerous mammalian skin cells that were not contacted with the drug.
